(19) United States
(12) Patent Application Publication
(10) Pub. No.: US 2019/0038717 A1
(43) Pub. Date: Feb. 7, 2019

(54) MESENCHYMAL STEM CELL EXPRESSING HEPATOCYTE GROWTH FACTOR, AND USE THEREOF

(71) Applicant: SLBIGEN INC., Seongnam-si, Gyeonggi-do (KR)

(72) Inventors: Young Chul SUNG, Seoul (KR); Soon Min LEE, Seoul (KR); Hey-yon KIM, Seoul (KR)

(73) Assignee: SLBIGEN INC., Seongnam-si, Gyeonggi-do (KR)

(21) Appl. No.: 16/075,457

(22) PCT Filed: Feb. 6, 2017
(86) PCT No.: PCT/KR2017/001301

§ 371 Date: Aug. 3, 2018

(30) Foreign Application Priority Data
Feb. 4, 2016 (KR) 10-2016-0014454

(51) Int. Cl. A61K 38/18; A61P 9/10; C12N 15/86; C12N 5/0775

(52) U.S. Cl. CPC A61K 38/1833; A61P 9/10; C12N 2760/18541; C12N 5/0663; C12N 2710/16141; C12N 15/86

(57) ABSTRACT

A recombinant lentiviral vector includes a gene encoding a hepatocyte growth factor (HGF) protein. And a cell that is transfected with the lentivirus produced by using the vector is provided. The recombinant lentivirus includes a gene encoding a HGF protein, and a host cell transfected with the lentivirus maintains a high cell proliferation rate. Thus, a mesenchymal stem cell expressing HGF by being transfected with the lentivirus may be usefully employed as a cell therapeutic agent.

### TECHNICAL FIELD

The present invention relates to a recombinant lentiviral vector comprising a gene encoding a hepatocyte growth factor (HGF) protein, and a cell transfected with the lentivirus produced by using the vector.

### BACKGROUND ART

Therapies utilizing cells are being developed globally, and especially, the stem cell therapy market is showing a steady upward trend with an annual average growth rate of 11.7%.

Mesenchymal stem cells (MSCs), which are adult stem cells, are multipotent cells that can differentiate into bones, cartilages, muscles, fats, and fibroblasts. In addition, the MSCs can be obtained from various adult tissues such as bone marrow, umbilical cord blood, and fats, relatively easily. MSCs are characterized by their ability to migrate to the site of inflammation or injury, which is also a great advantage as a delivery vehicle for delivering a therapeutic drug. In addition, the immune function of the human body can be regulated by inhibiting the functions of immune cells such as T cells, B cells, dendritic cells, and natural killer cells. Additionally, MSCs have an advantage that it can be cultured relatively easily in vitro, and thus studies for using MSCs as a cell therapeutic agent are being actively carried out.

However, despite such advantages of MSCs, there are following problems in producing MSCs that can be used clinically as a cell therapeutic agent. First, since there is a limitation in the proliferation of MSCs, it is difficult to produce them in large quantities. Second, since the MSCs obtained are heterogenous, it is difficult to maintain the same effect in every production. Third, the use of MSCs alone is not effective.

On the other hand, Korean Patent No. 1585032 discloses a cell therapeutic agent containing mesenchymal stem cells cultured in a hydrogel. The above document provides a composition that can be administered directly by shortening the pretreatment process in the step of isolating mesenchymal stem cells for use as a cell therapeutic agent. However, the problems of the mesenchymal stem cells described above and the method for solving the problems are not mentioned at all. Therefore, it is necessary to study mesenchymal stem cells which can be useful as a cell therapeutic agent.

### DISCLOSURE OF INVENTION

### Technical Problem

An object of the present invention is to provide a recombinant lentivirus comprising a gene encoding a HGF protein and a host cell transfected with the above recombinant lentivirus.

Another object of the present invention is to provide a pharmaceutical composition comprising the above recombinant lentivirus or host cell.

### Solution to Problem

In accordance with one object of the present invention, there is provided a recombinant lentiviral vector comprising a gene encoding a HGF protein.

Further, in accordance with another object of the present invention, there is provided a recombinant lentivirus comprising a gene encoding a HGF protein.

Further, in accordance with another object of the present invention, there is provided a host cell transfected with the above recombinant lentivirus.

Further, in accordance with another object of the present invention, there is provided a pharmaceutical composition for preventing or treating a vascular disease comprising the above recombinant lentivirus as an active ingredient.

Further, in accordance with another object of the present invention, there is provided a pharmaceutical composition for preventing or treating a vascular disease comprising the above host cell as an active ingredient.

### Advantageous Effects of Invention

A host cell transfected with a recombinant lentivirus comprising a gene encoding a HGF protein of the present invention expresses HGF and maintains a high cell proliferation rate. In addition, abnormal differentiation can be inhibited and the possibility of tumor formation can be blocked, indicating high safety. Therefore, the host cell expressing the HGF can be useful as a cell therapeutic agent.

### BRIEF DESCRIPTION OF DRAWINGS

FIG. 1 is a graph comparing cell proliferation rates of immortalized MSCs and non-immortalized MSCs:

imMSC: immortalized MSC;

MSC: non-immortalized MSC;

X axis: incubation period; and

Y axis: cumulative population doubling level (PDL).

FIG. 2 is a schematic representation of the structure of a gene construct inserted into a pBD-4 lentiviral vector:

TRE: a promoter comprising tetracycline response elements;

HGF: hepatocyte growth factor;

RSVp: RSV promoter; and

Hygro^{R}: a gene with resistance to hygromycin.

FIG. 3 is a graph showing the cell proliferation rate of immortalized MSCs transfected with lentiviruses containing the HGF gene:

X axis: incubation period; and

Y axis: cumulative PDL.

FIG. 4 shows the expression of HGF in BM-34A, a deposited strain. Lane 1 shows a marker, lanes 2 and 3 show BM-34A, lane 4 shows a negative control and lane 5 shows a positive control.

FIG. 5 is a graph showing the expression ratio of HGF protein in the BM-34A cell line at three different passages.

FIG. 6 is a graph showing the measured PDL of BM-34A cells obtained by subculture.

FIG. 7 shows the results of analysis of the karyotype of the gene introduced cell of the BM-34A cell line.

### BEST MODE FOR CARRYING OUT THE INVENTION

Hereinafter, the present invention will be described in detail.

The present invention provides a recombinant lentiviral vector comprising a gene encoding a HGF protein.

The term “hepatocyte growth factor (hereinafter, referred to as HGF)” protein, as used herein, is a heparin binding glycoprotein known as a scatter factor or hepatopoietin-A. It is produced by various mesenchymal cells and promotes cell proliferation. In addition, HGF is known to regulate the growth of endothelial cells and migration of vascular smooth muscle cells and induce angiogenesis.

A HGF protein according to the present invention may be a human-derived protein. The HGF protein is a heterodimer protein consisting of a 69 kDa α chain and a 34 kDa β chain, and may include four kringle structures in the α chain. The HGF protein of the present invention may be a polypeptide having the amino acid sequence of SEQ ID NO: 1. The HGF protein may have about 70%, 80%, 90%, 95% or higher homology with the amino acid sequence of SEQ ID NO: 1. Meanwhile, the gene encoding the HGF protein may be a polynucleotide having the nucleotide sequence of SEQ ID NO: 2. In addition, the nucleotide sequence encoding the HGF protein may have about 70%, 80%, 90%, 95% or higher homology with the nucleotide sequence of SEQ ID NO: 2.

The term “lentiviral vector” as used herein is a kind of retroviruses, which is a vector in the form of single stranded RNA, and may also be interchangeably referred to as a lentivirus transfer vector. The lentiviral vector can be inserted into the genomic DNA of a target cell of infection, to stably express the gene, and can transfer the gene to the mitotic and non-mitotic cells. Since the vector does not induce the immune response of a human body, its expression is continuous. In addition, there is an advantage that genes of a large size can be delivered as compared to an adenovirus vector which is a conventional virus vector.

The lentiviral vector may further comprise a gene encoding a thymidine kinase (TK) protein. The TK protein is an enzyme that catalyzes the thymidyl acid production reaction by binding phosphoric acid at the γ-position of ATP to thymidine, whereby thymidine is transformed into a triphosphate form. The modified thymidine cannot be used for DNA replication, and is known to induce death of cells containing it. The TK protein for use herein may be one of any known sequences. According to one embodiment, the TK protein may be a polypeptide having the amino acid sequence of SEQ ID NO: 3. Meanwhile, the gene encoding the TK protein may be a polynucleotide having the nucleotide sequence of SEQ ID NO: 4.

The recombinant lentiviral vector of the present invention can regulate the expression of a gene loaded thereto by a promoter. The promoter may be a cytomegalovirus (CMV), respiratory syncytial virus (RSV), human elongation factor-1 alpha (EF-1α) or tetracycline response elements (TRE) promoter. According to one embodiment, the recombinant lentiviral vector can regulate the expression of HGF protein by one promoter. The promoter is operably linked to a gene encoding a protein to be expressed.

According to one embodiment, the HGF protein may be linked to a TRE promoter. The TRE promoter can activate the transcription of the gene linked to the promoter by the tetracycline transactivator (tTA) protein. Specifically, the tTA protein binds to the TRE promoter and activates transcription when tetracycline or doxycycline is not present, whereas it cannot bind to the TRE promoter and activate the transcription when tetracycline or doxycycline is present. Thus, the expression of HGF protein can be regulated by the addition or depletion of tetracycline or doxycycline.

The term “operably linked” means that a particular polynucleotide is linked to another polynucleotide so that it can perform its function. The expression that a gene encoding a specific protein is operably linked to a promoter implies that it is linked such that the gene can be transcribed into mRNA by the action of the promoter and translated into a protein.

The present invention provides a recombinant lentivirus comprising a gene encoding a HGF protein.

The recombinant lentivirus may be obtained by the steps of transforming a host cell with a lentiviral vector of the present invention, a packaging plasmid and an envelope plasmid; and isolating the lentivirus from the transformed host cell.

The terms “packaging plasmid” and “envelope plasmid” may provide helper constructs (e.g., plasmids or separated nucleic acid) for producing lentiviruses from the lentiviral vectors of the present invention for effective transfection. Such constructs contain useful elements for preparing and packaging lentiviral vectors in host cells. The above elements include a structural protein such as a gag precursor; a processing protein such as a pol precursor; protease; coat protein; and expression and regulatory signal necessary to prepare proteins and produce lentiviral particles in the host cell, etc.

For production of the recombinant lentivirus, Lenti-X Lentiviral Expression System provided by Clontech Laboratories Inc., a packaging plasmid (e.g., pRSV-Rev, psPAX, pCl-VSVG, pNHP, etc.), or an envelope plasmid (e.g., pMD2.G, pLTR-G, pHEF-VSVG, etc.) provided by Addgene, can be used.

Further, the present invention provides a host cell transfected with the above recombinant lentivirus.

The term “transfection” refers to the delivery of a gene loaded in a recombinant lentiviral vector through viral infection.

A host cell according to the present invention may be a human embryonic stem cell (hES), a bone marrow stem cell (BMSC), a mesenchymal stem cell (MSC), a human neural stem cell (hNSCs), a limbal stem cell, or an oral mucosal epithelial cell. According to an embodiment of the present invention, the host cell may be a mesenchymal stem cell.

The term “mesenchymal stem cell (MSC)” refers to a multipotent stromal cell capable of differentiating into various cells including osteocytes, chondrocytes and adipocytes. Mesenchymal stem cells can differentiate into the cells of specific organs such as a bone, a cartilage, a fat, a tendon, a nervous tissue, fibroblasts and myocytes. These cells can be isolated or purified from adipose tissues, bone marrows, peripheral blood, umbilical cord blood, periosteum, dermis, mesodermal-derived tissues, and the like.

The host cell may be prepared by the following method:

1) primary infection of host cells with the lentiviruses comprising hTERT and c-myc genes;

2) secondary infection of the primary-infected host cells with the lentiviruses comprising a tTA gene; and

3) tertiary infection of the secondary-infected host cells with the lentiviruses comprising a HGF gene.

In the step 1), hTERT and c-myc are genes that immortalize host cells. Genes known as immortalizing genes other than hTERT and c-myc can also be used. According to one embodiment, the hTERT and c-myc proteins may be polypeptides having the amino acid sequences of SEQ ID NO: 7 and SEQ ID NO: 5, respectively. Meanwhile, the genes coding for the hTERT and c-myc proteins may be polynucleotides having the nucleotide sequences of SEQ ID NO: 8 and SEQ ID NO: 6, respectively.

In the step 2), tTA is a gene capable of regulating the expression of a target protein, which means tetracycline transactivator. The Tet-off system as used herein can regulate the expression of a target protein depending on the presence or absence of tetracycline or doxycycline as described above.

According to one embodiment corresponding to the above step 3), the cells expressing the HGF gene were prepared and obtained by tertiary infection of the immortalized MSC with lentiviruses comprising the HGF gene. The prepared cells were designated as BM-34A and deposited on Jan. 6, 2017 with the deposit number KCTC 13183BP at Korean Collection for Type Cultures at Korea Research Institute of Bioscience & Biotechnology (KRIBB).

The present invention provides a pharmaceutical composition for preventing or treating a vascular disease, comprising the above recombinant lentivirus or host cell as an active ingredient.

The term “vascular disease” as used herein refers to a disease that may be caused by aging or loss of elasticity of blood vessels. The recombinant lentivirus or host cell of the present invention can exhibit an angiogenic effect through the expression of HGF, and thus can be used for the treatment of various vascular diseases.

The vascular disease is a disease of a coronary artery, a cerebral blood vessel, a peripheral artery disease, or the like, which may be selected from the group consisting of angina pectoris, myocardial infarction, arteriosclerosis, atherosclerosis, periarteritis nodosa, Takayasu's arteritis, vascular occlusion, stroke, cerebral hemorrhage, cerebral infarction, cerebral edema and ischemic diseases.

The pharmaceutical composition is a kind of cell therapeutic agents, and may further comprise a pharmaceutically acceptable carrier, an additive or an excipient necessary for the formulation of the pharmaceutical composition. The carrier may be one generally used in the preparation of medicines, which may include lactose, dextrose, sucrose, sorbitol, mannitol, starches, gum acacia, calcium phosphate, alginates, gelatin, calcium silicate, microcrystalline cellulose, polyvinylpyrrolidone, cellulose, water, syrup, methylcellulose, methylhydroxybenzoate, propylhydroxybenzoate, talc, magnesium stearate, mineral oil, and the like.

In addition, the pharmaceutical compositions of the present invention may further comprise a pharmaceutically acceptable additive, which be selected from the group consisting of a lubricant, a wetting agent, a sweetening agent, a flavoring agent, an emulsifying agent, a suspending agent, a preservative and a combination thereof.

The carrier may be comprised in an amount of about 1% to about 99.99% by weight, preferably about 90% to about 99.99% by weight, based on the total weight of the pharmaceutical composition of the present invention, and the pharmaceutically acceptable additive may be comprised in an amount of about 0.1% to about 20% by weight.

The pharmaceutical composition may be prepared in a unit dosage form by formuling with a pharmaceutically acceptable carrier and excipient according to a conventional method, or may be prepared by filling into a multi-dose container. Herein, the formulation may be in the form of a solution, a suspension, a syrup or an emulsion in oil or aqueous media, or in the form of an extract, powders, a powdered drug, granules, tablets or capsules, and may additionally contain a dispersing or stabilizing agent.

The present invention also provides a method for preventing or treating a vascular disease as described above, comprising the step of administering a pharmaceutical composition of the present invention to a subject.

The subject may be a mammal, particularly a human. The administration route and dosage of the pharmaceutical composition may be adjusted in various ways and amounts for administration to a subject depending on the condition of a patient and the presence of side effects, and the optimal administration method and dosage may be selected by a person skilled in the art in a suitable range. In addition, the pharmaceutical composition may be administered in combination with other drugs or physiologically active substances known to have a therapeutic effect on a disease to be treated, or may be formulated in a form of combination formulation with other drugs.

When the pharmaceutical composition is administered parenterally, examples of the administration include subcutaneous, ocular, intraperitoneal, intramuscular, oral, rectal, intraorbital, intracerebral, intracranial, intraspinal, intraventricular, intrathecal, intranasal, intravenous, intracardiac administration.

The above administration may be administered for one or more times, one to three times, specifically in two divided doses. In the case of repeated administrations, they can be administered at the interval of 12 to 48 hours, 24 to 36 hours, and more specifically, at the interval of 24 hours. In the case of lentiviruses, the administration may be conducted in an amount of 1.0×10⁶ to 1.0×10¹² TU, specifically 1.0×10⁸ to 1.0×10¹⁰ TU for adults. On the other hand, in the case of cells, the administration may be conducted in an amount of 1.0×10⁵ to 1.0×10¹¹ cells, specifically 1.0×10⁷ to 1.0×10⁹ cells for adults. When the dose is high, the administration may be conducted several times a day.

### MODE FOR THE INVENTION

Hereinafter, the present invention is described in more detail with reference to Examples. It will be apparent to those skilled in the art that these Examples are for illustrative purpose only, and the scope of the present invention is not intended to be limited by these Examples

### Example 1. Preparation of Immortalized Mesenchymal Stem Cells (MSCs)

1-1. Preparation of Lentiviral Vectors Containing Immortalized Gene

In order to immortalize MSCs, lentiviral vectors respectively containing c-Myc and hTERT, which are immortalized genes, were prepared. Herein, a gene construct expressing the tTA protein was inserted together to use the Tet-off system.

First, a pBD lentiviral vector was prepared by substituting the EF promoter in the expression cassette of the pWPT vector (Addgene, USA) with the CMV promoter, and adding the RSV promoter to the downstream thereof.

The c-Myc gene (SEQ ID NO: 6) and thymidine kinase (TK) gene (SEQ ID NO: 4) were linked via IRES and then inserted into the pBD lentiviral vector so that the expression can be regulated by the CMV promoter. The constructed vector was designated as pBD-1.

On the other hand, the hTERT gene (SEQ ID NO: 8) was inserted into the pBD lentiviral vector such that the expression can be regulated by the CMV promoter. A gene having resistance to zeomycin (ZeoR; SEQ ID NO: 14) was inserted thereto such that the expression can be regulated by the RSV promoter. The constructed vector was designated as pBD-2.

In addition, a tTA (tetracycline transactivator) gene (SEQ ID NO: 10) was inserted into the pBD lentiviral vector such that the expression can be regulated by the CMV promoter. A gene having resistance to puromycin (PuroR; SEQ ID NO: 12) was inserted thereto such that the expression can be regulated by the RSV promoter. The constructed vector was designated as pBD-3.

1-2. Production of Lentiviruses Containing Immortalized Gene

Using the lentiviral vectors constructed in Example 1-1, lentiviruses containing immortalized genes were produced by the following method.

First, Lenti-X cells (Clontech Laboratories, USA) were cultured in a 150 mm dish using DMEM supplemented with 10% fetal bovine serum. Meanwhile, lentiviral vectors were extracted and quantified from DH5a E. coli cells using EndoFree Plasmin Maxi Kit (Qiagen, USA).

The cultured Lenti-X cells were washed with PBS, and then 3 ml of TrypLE™ Select CTS™ (Gibco, USA) was added thereto. The cells were left at 37° C. for about 5 minutes, and then their detachment was verified. The detached cells were neutralized by adding 7 ml of DMEM supplemented with 10% fetal bovine serum thereto. The neutralized cells were collected in a 50 ml tube and centrifuged at 1,500 rpm for 5 minutes. The resultant supernatant was removed and the cells were resuspended by adding 10 ml of DMEM supplemented with 10% fetal bovine serum thereto. The suspended cells were counted with a hematocytometer and then dispensed into a 150 mm dish in an amount of 1.2×10⁷ cells. When the dispensed cells were cultured to a cell saturation of about 90%, the cells were transformed with a mixture of 12 μg of lentiviral vectors, 12 μg of psPAX (Addgene; gag-pol expressing, packaging plasmid) and 2.4 μg of pMD.G plasmid (Addgene; VSVG expressing, envelope plasmid). In order to facilitate the transformation, lipofectamine (Invitrogen, USA) and PLUS reagent (Invitrogen, USA) were used. 6 hours after the transformation, the medium was replaced with DMEM supplemented with 10% fetal bovine serum. After 48 hours of additional culturing, the supernatant was collected.

The obtained supernatant was mixed with a lentivirus concentration kit (Lenti-X concentrator, Clontech Laboratories, USA) and then cultured overnight at 4° C. It was centrifuged under the condition of 4° C. and 4,000 rpm for 2 hours to obtain viruses, which were then resuspended in 0.5 ml of DMEM not containing FBS. As a result, lentiviruses produced from pBD-1, pBD-2 and pBD-3 lentiviral vectors were prepared at the concentrations of 4.0×10⁸ TU/ml, 2.0×10⁸ TU/ml and 1.2×10⁹ TU/ml, respectively.

1-3. Preparation of Immortalized Mesenchymal Stem Cells

Immortalized MSCs were prepared using the lentiviruses containing the immortalized genes produced in Example 1-2.

First, bone marrow-derived MSCs were prepared by the following method. Specifically, bone marrow aspirate was obtained from the iliac crest of a healthy donor. The aspirate was mixed with 20 IU/ml heparin in a sterile container to inhibit coagulation. The bone marrow mixture solution was centrifuged under the condition of 4° C., 739 g for 7 minutes, and then the supernatant was removed and the resultant was mixed with 10-fold amount (in volume) of sterilized water. The resultant mixture was centrifuged again under the same condition to obtain a cell pellet. The obtained pellet was suspended in DMEM-low glucose (11885-084, Gibco, USA) supplemented with 20% FBS and 5 ng/ml b-FGF (100-18B, Peprotech, USA), which was then dispensed into a culture flask. It was cultured under the condition of 37° C., 5% CO₂ for 24 to 48 hours, and then replaced with a new medium. The cells were cultured with passages while the medium was replaced with new medium at the interval of 3 to 4 days. After 2 weeks of culturing, MSCs were confirmed using a fluorescent cell analyzer.

The MSCs prepared above were infected with the pBD-1 lentiviruses produced in Example 1-2 at 100 MOI using Retronectin (Clontech Laboratories, USA). The infected cells were infected with the pBD-2 lentiviral vector at 100 MOI by the same method. After infection, the cells infected with pBD-2 lentiviruses were selected by adding 500 μg/ml zeomycin to the culture medium of the stabilized cells.

The selected cells were infected with pBD-3 lentiviral vector at 100 MOI. After infection, the cells infected with pBD-3 lentiviruses were selected by adding 1 μg/ml puromycin to the culture medium of the stabilized cells.

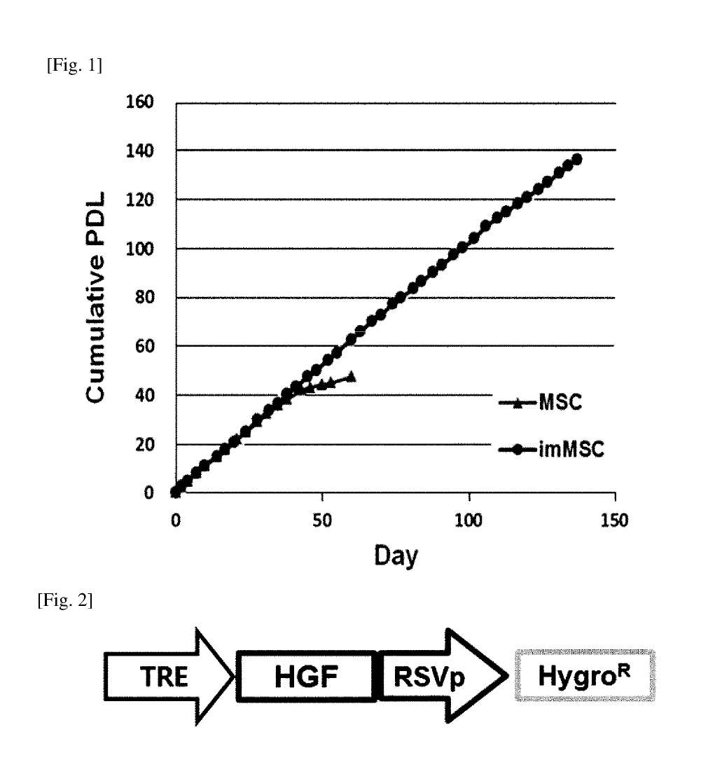

As a result, the cell proliferation rates of the MSCs containing the immortalized gene and the MSCs not containing the immortalized gene are shown in FIG. 1. As shown in FIG. 1, the MSCs infected with lentiviruses containing the immortalized genes, c-myc and hTERT, maintained high cell proliferation rates even after 120 days of culture. On the other hand, the cell proliferation rate of non-immortalized MSCs (MSC) decreased rapidly after 40 days of culture.

### Example 2. Construction of Lentivirus Containing HGF Gene

2-1. Construction of Lentiviral Vector Containing HGF Gene

The HGF gene (SEQ ID NO: 2) was inserted into the pBD lentiviral vector produced as above. Herein, the inserted HGF gene was designed to be regulated by the TRE promoter. The TRE promoter can regulate the expression of a gene linked thereto depending on the presence or absence of the addition of doxycycline.

Herein, a gene having hygromycin resistance (HygroR; SEQ ID NO: 16) was inserted such that its expression can be regulated by the RSV promoter. The constructed vector was designated as pBD-4, and the structure of the gene construct is shown in FIG. 2.

2-2. Production of Lentivirus Containing HGF Gene

Using the lentiviral vector containing the HGF gene constructed in Example 2-1, lentivirus was produced by the same method as described in Example 1-2. The lentivirus produced was prepared at a concentration of 3.5×10⁸ TU/ml.

### Example 3 Preparation of MSC Infected with Lentivirus Containing HGF Gene

3-1. Preparation of MSC Transfected with Lentivirus Containing HGF Gene

Cells expressing the HGF gene were prepared by infecting the immortalized MSC prepared in Example 1-3 with the lentiviruses containing the HGF gene constructed in Example 2-2. Infection was carried out by the same method as described in Example 1-3. After infection, the cells infected with pBD-4 lentiviruses were selected by adding 25 μg/ml hygromycin to the culture medium of the stabilized cells. The selected cells were cultured in a medium supplemented with 2 μg/ml of doxycycline (631311, Clontech, USA), thereby suppressing the expression of the HGF protein during the culture.

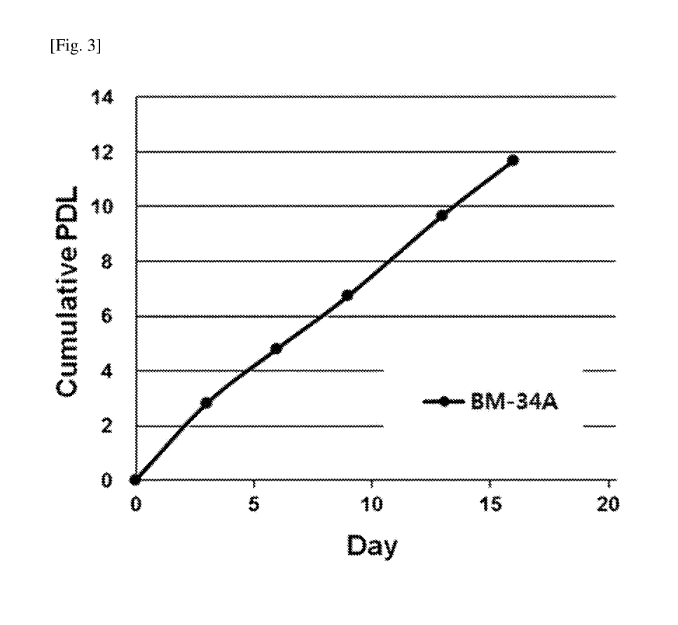

The selected cells were cultured to form colonies. The monoclonal cells obtained from the colonies formed were cultured to establish a cell line, which was designated as BM-34A. The cell line BM-34A was deposited on Jan. 6, 2017 with the deposit number KCTC 13183BP at Korean Collection for Type Cultures at Korea Research Institute of Bioscience & Biotechnology (KRIBB). As a result, the proliferation rate of established cell line is shown in FIG. 3. It shows that the BM-34A cell line stably proliferated.

3-2. Test for Examination of Introduced Gene of BM-34A Cell Line

A sample of the established cell line, BM-34A, was thawed for about 1 minute in a constant temperature water bath at 37° C., transferred to a 15 ml tube containing 9 ml PBS, and subjected to a Cell Down process for 5 minutes at 1,500 rpm. After PBS was completely removed, the pellet was suspended in 200 μl of PBS, and transferred to in a 1.5 ml tube. gDNA was prepared using NucleoSpin® Tissue (MN, 740952.250), and the mixture was prepared as shown in Table 1, followed by PCR by the steps shown in Table 2 below. Herein, 100 ng of BM-34A plasmid DNA was added as a positive control and 1 μl of dW was used as a negative control.

TABLE 1

| | |
|---|---|
| Forward primer (SEQ ID NO: 17) (10 pmol/μl, | 1 μl |
| BM163) | |
| Reverse primer (SEQ ID NO: 18) (10 pmol/μl, | 1 μl |
| BM151) | |
| Sample (100 ng/μl) | 1 μl |
| dW | 17 μl |
| Total volume | 20 μl |

TABLE 2

| Step | Temperature | Time | Cycle |
|---|---|---|---|
| 1st | 95° C. | 5 min | 1 |
| 2nd | 95° C. | 45 sec | 35 |
| | 60° C. | 45 sec | |
| | 72° C. | 1 min | |
| 3rd | 72° C. | 7 min | 1 |
| 4th | 4° C. | Indefinitely | 1 |

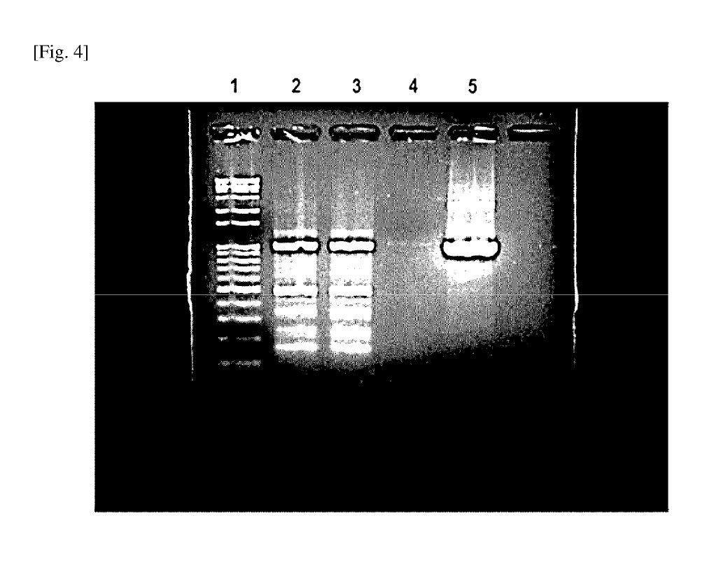

1% agarose gel was placed in an electrophoresis kit. 10 μl of DNA Size Marker was loaded in the first well and 10 μl each of BM-34A sample (×2), a negative control and a positive control were loaded in the following wells respectively in the above order. Thereafter, electrophoresis was conducted at 100 V for 20 minutes, and a gel photograph was taken. The result is shown in FIG. 4.

As shown in FIG. 4, both of the two BM-34A cell line samples showed PCR products of the same size (1.0 kb) as the positive control.

3-3. Identification of HGF Protein Expression in Established Cell Lines

HGF protein expression in the BM-34A cell line established in Example 3-1 was examined by ELISA analysis.

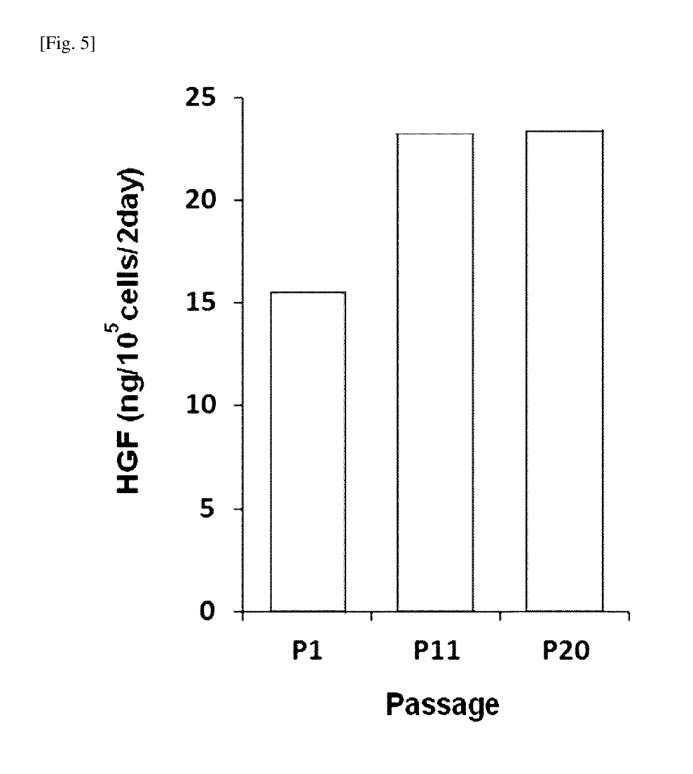

Specifically, the cells were cultured for two days in a culture medium not containing doxycycline. The BM-34A cell line was seeded on a 12-well plate at 1×10⁵ cells to a total volume of 1 ml. After 48 hours, approximately 1 ml of the cell culture medium was obtained and analyzed using a human HGF DuoSet ELISA kit (R&D systems, DY294, USA). Experiments were conducted according to the manual included in each kit. In order to examine whether there is no change in the expression rate for each passage, cells of three different passages were analyzed. The result of the analysis is shown in FIG. 5, and the levels of HGF protein expression during 24 hours induced in about 1×10⁵ cells in the medium from which doxycycline was removed are shown in Table 3 below.

TABLE 3

| Target protein | Expression level |
|---|---|
| HGF | 47.72 ng/ml |

As shown in FIG. 5, it was found that HGF was expressed in the BM-34A cell line cultured in the medium from which doxycycline was removed. As shown in Table 3, the HGF protein of about 47.72 ng/ml was expressed in the BM-34A cell line of the present invention.

3-4. Cell Population Doubling Level (PDL) Analysis

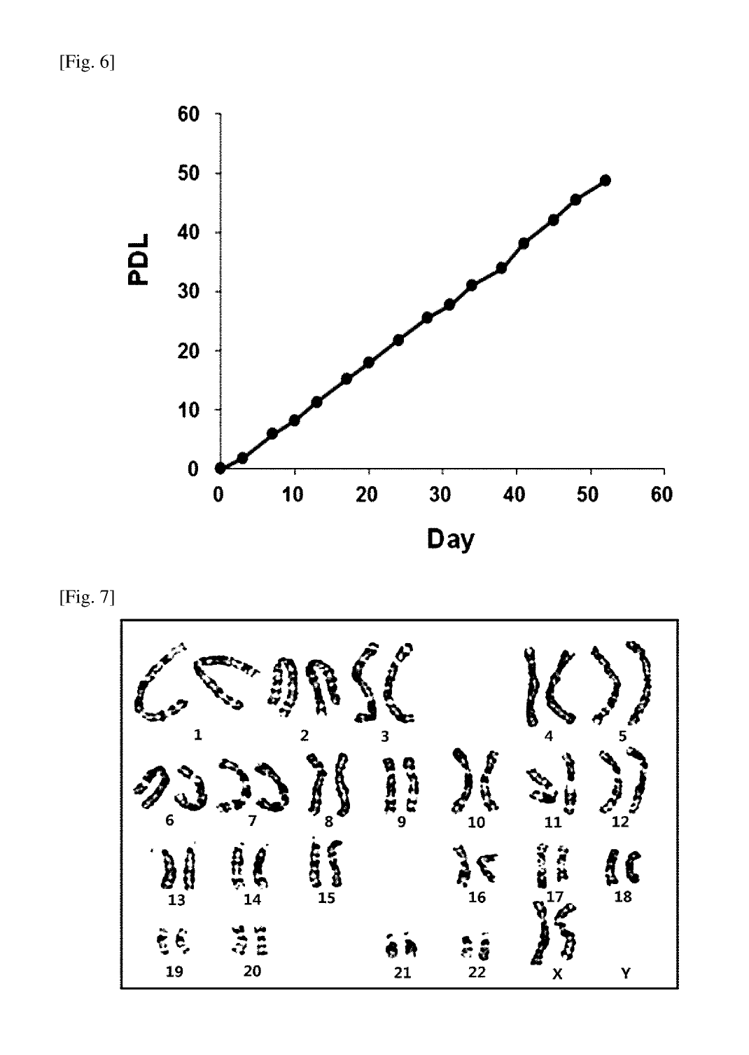

The BM-34A cell line was seeded in a T75 flask at 4×10⁵ cells using a medium containing 2 μg/ml of doxycycline. Cells were obtained through subculture for about 3 or 4 days, and the total number of cells was counted. Cells of the same number were seeded, and PDL was measured every 3 to 4 days. The PDL was calculated using the following Equation 1, and the result is shown in FIG. 6. In the Equation 1, X represents the initial PDL, I represents the initial number of cells seeded in the blood vessel, Y represents the final cell yield or the number of cells in the end of growth period.

PDL=X+3.222(log Y−log I) [Equation 1]

As shown in FIG. 6, stable growth was observed even in long-term subculture.

3-5. Karyotype Analysis of Cells

In order to examine the chromosomal abnormality of the cells to which genes were introduced into the BM-34A cell line, analysis was requested to EONE Life Science Institute (Korea) and conducted in accordance with the protocol. The result of the analysis is shown in FIG. 7.

As shown in FIG. 7, no abnormality was observed in the chromosome of the cells to which genes were introduced into the BM-34A cell line, and thus it was determined as a normal karyotype.

1. A recombinant lentiviral vector comprising a gene encoding a hepatocyte growth factor (HGF) protein.

2. The recombinant lentiviral vector according to claim 1, wherein the HGF protein is a polypeptide having the amino acid sequence of SEQ ID NO: 1.

3. The recombinant lentiviral vector according to claim 1, wherein the vector comprises a promoter.

4. The recombinant lentiviral vector according to claim 3, wherein the promoter is a cytomegalovirus (CMV), respiratory syncytial virus (RSV), human elongation factor-1 alpha (EF-1α) or tetracycline response elements (TRE) promoter.

5. A recombinant lentivirus comprising a gene encoding a HGF protein.

6. The recombinant lentivirus according to claim 5, wherein the lentivirus is obtained by the steps of:
transforming a host cell with the lentiviral vector of claim 1, a packaging plasmid and an envelope plasmid; and
isolating the lentivirus from the transformed host cell.

7. A host cell transfected with the recombinant lentivirus according to claim 5.

8. The host cell according to claim 7, which is a mesenchymal stem cell.

9. A pharmaceutical composition comprising the recombinant lentivirus according to claim 5 as an active ingredient.

10. (canceled)

11. A pharmaceutical composition comprising the host cell according to claim 7 as an active ingredient.

12. A method for preventing or treating a vascular disease of a subject in need thereof comprising administering a recombinant lentivirus or a host cell transfected with the recombinant lentivirus, as an active ingredient, to the subject,
wherein the recombinant lentivirus comprises the recombinant lentiviral vector of claim 1 encoding a hepatocyte growth factor (HGF) protein.

13. The method according to claim 12, wherein the vascular disease is selected from the group consisting of angina pectoris, myocardial infarction, arteriosclerosis, atherosclerosis, periarteritis nodosa, Takayasu's arteritis, vascular occlusion, stroke, cerebral hemorrhage, cerebral infarction, cerebral edema, and ischemic diseases.

14. The method of claim 12, wherein the host cell is mesenchymal stem cell.

15. The method of claim 12, wherein the HGF protein is a polypeptide having the amino acid sequence of SEQ ID NO: 1.

16. The method of claim 12, wherein the recombinant lentiviral vector comprises a plasmid selected from the group consisting of a cytomegalovirus (CMV), respiratory syncytial virus (RSV), human elongation factor-1 alpha (EF-1α) or tetracycline response elements (TRE) promoter.


SEQUENCE LISTING (18 sequences)

SEQ ID NO: 1 - protein, 728 aa, Artificial Sequence
  amino acid sequence of HGF
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 2 - DNA, 2187 bp, Artificial Sequence
  nucleotide sequence of HGF
atgtgggtgaccaagctgctgcccgccctgctgctgcagcacgtgctgctgcacctgctcctgctgcccatcgccatcccctacgccgagggccagagaaagagaagaaacaccatccacgagttcaagaagagcgccaagaccacactgatcaagatcgaccccgccctgaagatcaagaccaagaaggtgaacaccgccgaccagtgcgccaacagatgcaccagaaacaagggcctgcccttcacctgcaaggccttcgtgttcgacaaggccagaaagcagtgcctgtggttccccttcaacagcatgagcagcggcgtgaagaaggagttcggccacgagttcgacctgtacgagaacaaggactacatcagaaactgcatcatcggcaagggcagaagctacaagggcaccgtgagcatcaccaagagcggcatcaagtgccagccctggagcagcatgatcccccacgagcacagcttcctgcccagcagctacagaggcaaggacctgcaggagaactactgtagaaaccctagaggcgaggagggcggaccctggtgcttcaccagcaaccccgaggtgagatacgaggtgtgcgacatcccccagtgcagcgaggtggagtgcatgacctgcaacggcgagagctacagaggcctgatggaccacaccgagagcggcaagatctgccagagatgggaccaccagaccccccacagacacaagttcctgcccgagagataccccgacaagggcttcgacgacaattactgcaggaaccctgatggccagcccagaccctggtgctacaccctggacccccacaccagatgggagtactgcgccatcaagacctgcgccgacaacaccatgaacgacaccgacgtgcccctggagacaaccgagtgcatccagggccagggcgagggctacagaggcaccgtgaacaccatctggaacggcatcccctgccagaggtgggatagccagtacccccacgagcacgacatgacccccgagaacttcaagtgcaaggacctgagagagaattactgtaggaatcccgacggcagcgagagcccctggtgcttcacaaccgaccccaacatcagagtgggctactgcagccagatccccaactgcgacatgagccacggccaggactgctacagaggcaacggcaagaactacatgggcaacctgagccagaccagaagcggcctgacctgcagcatgtgggacaagaacatggaggacctgcacagacacatcttctgggagcccgacgccagcaagctgaacgagaactactgcagaaatcccgacgacgacgcccacggcccttggtgctacaccggcaaccccctgatcccctgggactactgccccatcagcagatgcgagggcgacaccacacccaccatcgtgaacctggaccaccccgtgatctcctgcgccaagaccaagcagctgagagtggtgaacggcatccccaccagaaccaacatcggctggatggtgagcctgagatacagaaacaagcacatctgcggcggcagcctgatcaaggagagctgggtgctgaccgccagacagtgcttccccagcagagacctgaaggactacgaggcctggctgggcatccacgacgtgcacggcagaggcgacgagaagtgcaagcaggtgctgaacgtgagccagctggtgtacggccccgagggcagcgacctggtgctgatgaagctggccagacccgccgtgctggacgacttcgtgagcaccatcgacctgcccaactacggctgcaccatccccgagaagaccagctgcagcgtgtacggctggggctacaccggcctgatcaactacgacggcctgctgagagtggcccacctgtacatcatgggcaacgagaagtgcagccagcaccacagaggcaaggtgaccctgaacgagagcgagatctgcgccggcgccgagaagatcggcagcggcccctgcgagggcgactacggcggccctctggtgtgcgagcagcacaagatgagaatggtgctgggcgtgatcgtgcccggcagaggctgcgccatccccaacagacccggcatcttcgtgagagtggcctactacgccaagtggattcacaagatcatcctgacctacaaggtgccccagagctaa

SEQ ID NO: 3 - protein, 376 aa, Artificial Sequence
  amino acid sequence of TK
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 4 - DNA, 1131 bp, Artificial Sequence
  nucleotide sequence of TK
atggccagctacccctgtcaccagcacgccagcgccttcgaccaggccgctagaagcagaggccacagcaacagaagaaccgccctgagacccagaagacagcaggaggccacagaggtgagactggagcagaagatgcccaccctgctgagagtgtacatcgatggaccccacggcatgggcaagaccacaacaacccagctgctggtggccctgggcagcagagacgacatcgtgtacgtgcccgagcccatgacctactggcaggtgctgggagccagcgagaccatcgccaacatctacaccacacagcacagactggaccagggcgagatcagcgccggcgacgctgccgtggtgatgaccagcgcccagatcacaatgggcatgccctacgccgtgaccgatgccgtgctggctccccacgtgggcggagaggccggcagcagccacgcccctccccctgccctgaccctgatcttcgacagacaccccatcgccgccctgctgtgctaccccgccgctagatacctgatgggcagcatgacaccccaggccgtgctggccttcgtggccctgatcccccctaccctgcccggcaccaacatcgtgctgggcgccctgcccgaggacagacacatcgacagactggctaagagacagagacccggcgagagactggacctggccatgctggccgccatcagaagagtgtacggcctgctggccaacaccgtgagatacctgcagggaggcggcagctggtgggaggactggggccagctgagcggcaccgccgtgcctccccagggcgccgagccccagagcaacgccggccctagaccccacatcggcgacaccctgttcaccctgtttagagcccccgagctgctggcccccaacggcgacctgtacaacgtgttcgcctgggccctggacgtgctggccaagagactgagacccatgcacgtgttcatcctggactacgaccagagccccgccggctgcagagatgccctgctgcagctgaccagcggcatggtgcagacccacgtgaccacacccggcagcatccccaccatctgcgacctggccagaacctttgccagagagatgggcgaggccaactga

SEQ ID NO: 5 - protein, 454 aa, Artificial Sequence
  amino acid sequence of c-Myc
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 6 - DNA, 1365 bp, Artificial Sequence
  nucleotide sequence of c-Myc
atggatttctttcgcgtcgtcgagaaccagcagccacccgccactatgcctctgaacgtgtcttttactaacaggaactatgatctggattacgacagcgtgcagccctacttctattgcgatgaggaagagaacttttatcagcagcagcagcagagcgagctgcagccacctgcaccttccgaagacatttggaagaaattcgagctgctgcctacaccacccctgtctccaagtcggagaagcggcctgtgttcacccagctacgtggccgtcactcctttcagcctgaggggggacaatgatggcgggggaggctccttttctacagccgatcagctggaaatggtgactgagctgctggggggagacatggtcaaccagagcttcatttgcgatcctgacgatgaaacttttatcaagaacatcatcatccaggactgtatgtggtcaggctttagcgccgctgcaaagctggtgtctgagaaactggcaagttatcaggccgctcggaaagatagtgggtcacctaacccagctagaggacactccgtgtgctctacaagctccctgtacctgcaggacctgagcgcagccgcttccgagtgtattgatccctccgtggtcttcccctatcctctgaatgactctagttcacccaagagttgcgcatcacaggacagctccgccttttcaccttctagtgatagcctgctgtcaagcactgaatcctctccacagggcagcccagagccactggtgctgcatgaagagacccctccaaccacaagttcagattccgaagaggaacaggaggacgaggaagagatcgatgtggtctctgtggaaaagcgccaggctccaggaaaacgaagcgagtccggctctccaagtgcaggaggacactccaagccacctcattctcccctggtgctgaaaaggtgccacgtctccacccaccagcataactacgcagccccaccctctacacgaaaggactatccagctgcaaagcgcgtgaaactggatagcgtgagagtcctgaggcagatcagtaacaatcggaagtgtacttcacccagaagctccgacaccgaagagaacgtgaaaaggcgcacccataatgtcctggaacgccagcgacggaatgagctgaagaggtccttctttgccctgcgcgatcagattcctgaactggagaacaatgagaaggctccaaaagtggtcattctgaagaaagccacagcttacatcctgtctgtgcaggccgaagagcagaaactgatcagtgaagaggacctgctgagaaaacgcagggaacagctgaaacataaactggaacagctgagaaactcttgtgcttaa

SEQ ID NO: 7 - protein, 1132 aa, Artificial Sequence
  amino acid sequence of hTERT
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 8 - DNA, 3399 bp, Artificial Sequence
  nucleotide sequence of hTERT
atgcccagagctcccagatgcagagccgtgagaagcctgctgagaagccactacagagaggtgctgcccctggccaccttcgtgagaagactgggaccccagggctggagactggtgcagagaggcgaccccgcagcctttagagccctggtggcccagtgcctggtgtgcgtgccctgggacgccagacctcctcccgctgcccccagcttcagacaggtgagctgcctgaaggagctggtggccagagtgctccagagactgtgcgagagaggcgccaagaacgtgctggcctttggcttcgccctgctggatggagccagaggcggacctcccgaggccttcaccacaagcgtgagaagctacctgcccaacaccgtgaccgatgccctgagaggctccggcgcctggggcctgctcctgagaagagtgggcgacgacgtgctggtgcacctgctggccagatgcgccctgttcgtgctggtggctcccagctgcgcctaccaggtgtgcggaccccctctgtaccagctgggagccgccacccaggcaagaccccctccccacgcctctggacccagaagaagactgggctgcgagagagcctggaaccacagcgtgagagaggctggcgtgcccctgggcctgcccgcccctggcgccagaagaagaggcggcagcgccagcagaagcctgcccctgcccaagagacccagacgcggcgccgctcccgagcctgagagaacacccgtgggccagggcagctgggcccaccccggcagaaccagaggacccagcgacagaggcttctgcgtggtgagccctgccagacccgccgaggaggccaccagcctggagggcgccctgagcggcaccagacacagccaccccagcgtgggcagacagcaccacgccggccctcctagcaccagcagaccccccagaccttgggacaccccctgcccccctgtgtacgccgagaccaagcacttcctgtacagcagcggcgacaaggagcagctgagacccagcttcctgctgagctccctgagacccagcctgaccggcgccagaagactggtggagaccatcttcctgggcagcagaccctggatgcccggcacccccagaagactgcccagactgccccagagatactggcagatgagacccctgttcctggagctgctgggcaaccacgcccagtgcccctacggcgtgctgctgaagacccactgccccctgagagctgccgtgacccccgcagctggcgtgtgcgccagagagaagccccagggcagcgtggccgctcccgaggaggaggacaccgatcccagaagactggtgcagctgctgagacagcacagcagcccctggcaggtgtacggcttcgtgagagcctgcctgagaagactggtgcctcccggcctgtggggcagcagacacaacgagagaagattcctgagaaacaccaagaagttcatcagcctgggcaagcacgccaagctgagcctccaggagctgacatggaagatgagcgtgagagactgcgcctggctgaggagaagccctggcgtgggctgcgtgcccgccgccgagcacagactgagagaggagatcctggccaagtttctgcactggctgatgagcgtgtacgtggtggagctgctgagaagcttcttctacgtgaccgagaccacattccagaagaacagactgttcttttacaggaagagcgtgtggagcaagctccagagcatcggcatcagacagcacctgaagagagtgcagctgagagagctgagcgaggccgaggtgagacagcacagagaggccagacccgccctgctgaccagcagactgagattcatccccaagcccgatggcctgagacccatcgtgaacatggactacgtggtgggagccagaacctttagaagagagaagagagccgagagactgaccagcagagtgaaggccctgttcagcgtgctgaactacgagagagccagaagacccggcctgctgggcgccagcgtgctgggcctggacgacatccacagagcctggagaaccttcgtgctgagagtgagagcccaggaccctcctcccgagctgtacttcgtgaaggtggacgtgaccggcgcctacgacaccatcccccaggacagactgaccgaggtgatcgccagcatcatcaagccccagaacacctactgcgtgagaagatacgccgtggtgcagaaggccgcccacggccacgtgagaaaggccttcaagagccacgtgagcaccctgaccgacctccagccctacatgagacagttcgtggcccacctccaggagaccagccccctgagagatgccgtggtgatcgagcagagctcttccctgaacgaggcctccagcggcctgttcgacgtgttcctgagattcatgtgccaccacgccgtgagaatcagaggcaagagctacgtgcagtgccagggcatcccccagggcagcatcctgagcaccctgctgtgcagcctgtgctacggcgacatggagaacaagctgttcgctggcatcagaagagacggcctgctgctgagactggtggacgacttcctgctggtgaccccccacctgacccacgccaagaccttcctgagaaccctggtgagaggcgtgcccgagtacggctgcgtggtgaacctgagaaagaccgtggtgaactttcccgtggaggacgaggccctgggcggcaccgccttcgtgcagatgcccgcccacggcctgtttccctggtgcggcctgctcctcgacaccagaaccctggaggtgcagagcgactacagcagctacgcaagaaccagcatcagagccagcctgaccttcaacagaggcttcaaggccggcagaaacatgagaagaaagctgttcggcgtgctgagactgaagtgccacagcctgttcctggacctccaggtgaacagcctccagaccgtgtgcaccaacatctacaagatcctgctgctccaggcctacagattccacgcctgcgtgctccagctgcccttccaccagcaggtgtggaagaatcccaccttcttcctgagagtgatcagcgacaccgccagcctgtgctacagcatcctgaaggccaagaatgccggcatgagcctgggcgccaagggcgccgctggacccctgcccagcgaggccgtgcagtggctgtgccaccaggccttcctgctgaagctgaccagacacagagtgacctacgtgcccctgctgggcagcctgagaaccgcccagacccagctgagcagaaagctgcctggcacaaccctgaccgccctggaggcagccgcaaaccccgccctgcccagcgacttcaagaccatcctggactag

SEQ ID NO: 9 - protein, 248 aa, Artificial Sequence
  amino acid sequence of tTA
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 10 - DNA, 747 bp, Artificial Sequence
  nucleotide sequence of tTA
atgtcaaggctggataaaagcaaagtgattaactccgctctggaactgctgaacgaagtcggcattgaggggctgaccacacgcaagctggcacagaagctgggagtggagcagcccaccctgtactggcacgtgaagaacaagcgcgccctgctggacgccctggccatcgagatgctggatcggcaccacacacacttctgccctctggagggcgagagctggcaggacttcctgcggaacaatgccaagagctttagatgtgccctgctgtcccacagggatggagcaaaggtgcacctgggcaccagaccaacagagaagcagtacgagaccctggagaaccagctggccttcctgtgccagcagggcttttctctggagaatgccctgtatgccctgagcgccgtgggacacttcaccctgggatgcgtgctggaggaccaggagcaccaggtggccaaggaggagagagagacacctaccacagactccatgccccctctgctgaggcaggccatcgagctgtttgatcaccagggcgccgagccagccttcctgtttggcctggagctgatcatctgcggcctggagaagcagctgaagtgtgagtctggaggaccagcagatgccctggacgatttcgacctggatatgctgcccgccgacgccctggacgattttgatctggacatgctgcctgctgatgccctggatgattttgacctggatatgctgcctggataa

SEQ ID NO: 11 - protein, 199 aa, Artificial Sequence
  amino acid sequence of PuroR
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 12 - DNA, 600 bp, Artificial Sequence
  nucleotide sequence of PuroR
atgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 13 - protein, 124 aa, Artificial Sequence
  amino acid sequence of ZeoR
MAKLTSAVPVLTARDVAGAVEFWTDRLGFSRDFVEDDFAGVVRDDVTLFISAVQDQVVPDNTLAWVWVRGLDELYAEWSEVVSTNFRDASGPAMTEIGEQPWGREFALRDPAGNCVHFVAEEQD

SEQ ID NO: 14 - DNA, 375 bp, Artificial Sequence
  nucleotide sequence of ZeoR
atggccaagttgaccagtgccgttccggtgctcaccgcgcgcgatgtggccggagcggtcgagttctggaccgaccggctcgggttcagccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactaa

SEQ ID NO: 15 - protein, 345 aa, Artificial Sequence
  amino acid sequence of HygroR
MKKPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKEVGRV

SEQ ID NO: 16 - DNA, 1038 bp, Artificial Sequence
  nucleotide sequence of HygroR
atgaagaagcccgaactcaccgctaccagcgttgaaaaatttctcatcgagaagttcgacagtgtgagcgacctgatgcagttgtcggagggcgaagagagccgagccttcagcttcgatgtcggcggacgcggctatgtactgcgggtgaatagctgcgctgatggcttctacaaagaccgctacgtgtaccgccacttcgccagcgctgcactacccatccccgaagtgttggacatcggcgagttcagcgagagcctgacatactgcatcagtagacgcgcccaaggcgttactctccaagacctccccgaaacagagctgcctgctgtgttacagcctgtcgccgaagctatggatgctattgccgccgccgacctcagtcaaaccagcggcttcggcccattcgggccccaaggcatcggccagtacacaacctggcgggatttcatttgcgccattgctgatccccatgtctaccactggcagaccgtgatggacgacaccgtgtccgccagcgtagctcaagccctggacgaactgatgctgtgggccgaagactgtcccgaggtgcgccacctcgtccatgccgacttcggcagcaacaacgtcctgaccgacaacggccgcatcaccgccgtaatcgactggtccgaagctatgttcggggacagtcagtacgaggtggccaacatcttcttctggcggccctggctggcttgcatggagcagcagactcgctacttcgagcgccggcatcccgagctggccggcagccctcgtctgcgagcctacatgctgcgcatcggcctggatcagctctaccagagcctcgtggacggcaacttcgacgatgctgcctgggctcaaggccgctgcgatgccatcgtccgcagcggggccggcaccgtcggtcgcacacaaatcgctcgccggagcgcagccgtatggaccgacggctgcgtcgaggtgctggccgacagcggcaaccgccggcccagtacacgaccgcgcgctaaggaggtaggtcgagtttaa

SEQ ID NO: 17 - DNA, 20 bp, Artificial Sequence
  BM163 Forward primer
tacatcatgggcaacgagaa

SEQ ID NO: 18 - DNA, 20 bp, Artificial Sequence
  BM151 Reverse primer
tcgtcctgcagttcattcag